(19) United States
(12) Patent Application Publication
(10) Pub. No.: US 2023/0107699 A1
(43) Pub. Date: Apr. 6, 2023

(54) DEVICE WITH AT LEAST ONE ELECTRODE UNIT FOR AN ELECTROSTIMULATION OR A DATA ACQUISITION BY DIAGNOSTIC INSTRUMENTS

(71) Applicant: Pierenkemper GmbH, Ehringshausen (DE)

(72) Inventors: Roger PIERENKEMPER, Marburg (DE); Jonas PEINZE, Nendeln (LI)

(73) Assignee: Pierenkemper GmbH, Ehringshausen (DE)

(21) Appl. No.: 17/953,604

(22) Filed: Sep. 27, 2022

(30) Foreign Application Priority Data
Oct. 1, 2021 (EP) 21200416.2

(51) Int. Cl. A61B 5/27; A61B 5/00; B32B 7/12; B32B 7/09; B32B 5/02; B32B 27/12; B32B 5/18; B32B 27/28

(52) U.S. Cl. CPC A61B 5/27; A61B 5/6804; B32B 7/12; B32B 7/09; B32B 5/026; B32B 27/12; B32B 5/022; B32B 5/18; B32B 27/283; B32B 2262/0276; B32B 2307/202; B32B 2437/00

(57) ABSTRACT

A device includes at least one electrode unit for an electrostimulation or a data acquisition by diagnostic instruments. The device is formed from a textile material and has at least two layers. The at least one electrode unit is disposed in or on a first layer, and a second layer is disposed on the first layer in such a way that the first layer and the second layer form at least one pocket. At least one support-pad part is disposed exchangeably in the pocket.

### CROSS REFERENCE TO RELATED APPLICATIONS

Applicant claims priority under 35 U.S.C. § 119 of European Application No. 21200416.2 filed Oct. 1, 2021, the disclosure of which is incorporated by reference.

### BACKGROUND OF THE INVENTION

### 1. Field of the Invention

The invention relates to a device with at least one electrode unit for an electrostimulation or a data acquisition by diagnostic instruments.

### 2. Description of the Related Art

A device for complex electromyostimulation belongs to the prior art (DE 20 2012 102 393 U1). This prior art device comprises a suit, a stimulation unit, an operator-control unit as well as a communication unit connecting the operator-control unit and the stimulation unit for signal transmission. This suit consists of a warp-knitted fabric. In this suit, the electrode unit is disposed on the suit. The electrodes consist of metallized polymer fibers. The contact electrodes may also consist of conductive polymer masses. These contact electrodes belonging to the prior art have the disadvantage that the polymer masses easily become brittle, especially during intensive use of the suit. If the polymer masses become brittle, the electrodes are no longer functional and the suit can no longer be used for the electrostimulation. In addition, the materials currently available on the market do not meet the strict cytotoxicity requirements imposed on products of this type.

Furthermore, an electrode arrangement and device for the treatment of pains belongs to the prior art (EP 2 815 787 A2).

This electrode arrangement belonging to the prior art has a layer structure, which consists of a carrier element, which may consist, for example, of knitted fabric or textile, and of a compression member disposed tightly in the device.

According to this prior art, the positions of the electrodes of the electrode array may be chosen such that this array corresponds to the shape of the body on which the electrode array is supposed to make contact. It is possible that the distribution of the electrodes of the electrode array follows the shape of a compression member provided tightly for exertion of a pressure. For example, different but tightly disposed compression members may be provided for different positions on the body. For example, compression members shaped as arch supports are known for a knee or a shoulder joint. The electrodes of the electrode array may be distributed spatially over the area of the compression member.

This device belonging to the prior art may be further improved by individually adjusting the pressing force of the electrode arrangement on the body of a wearer.

Despite that improvement, the compression members disposed tightly in the suits is a disadvantage when the suit is cleaned, because the compression members dry poorly.

Furthermore, a textile electrode device that likewise has a layer structure belongs to the prior art (WO 2009/043196 A1).

These electrodes integrated in textile, belonging to the prior art, for a transcutaneous electrostimulation (surface electrostimulation) are integrated in cuffs or clothing items for the application of electrotherapies, muscular buildup training, electrical pain therapy or functional electrostimulation.

According to this prior art, compression members are provided that are designed as inflatable compression members or as actuators. These compression members are of very complex structure and make the textile electrode device considerably more expensive, because a multiplicity of compression members is disposed in such a device, namely at every point at which an electrode arrangement is provided.

### SUMMARY OF THE INVENTION

The technical problem underlying the invention consists in specifying a device, with at least one electrode unit, for an electrostimulation or for a data acquisition by diagnostic instruments, which device permits an individual adaptation of the pressing pressure of the electrodes, which also is inexpensive and which is easy to wash and to sterilize.

This technical problem is solved by a device having the features according to the invention.

The device according to the invention with at least one electrode unit for an electrostimulation or a data acquisition by diagnostic instruments, wherein the device is formed from a textile material, wherein the device is formed from at least two layers, wherein the at least one electrode unit is disposed in or on a first layer and a second layer is disposed on the first layer in such a way that the first and the second layer form at least one pocket is characterized in that at least one support-pad part is disposed exchangeably in the pocket.

Because the at least one support-pad part is disposed exchangeably in the pocket, it is possible to use support-pad parts that generate the necessary pressing pressure of the electrode unit on the body of the wearer of the device.

Thus, it is possible, for example, when the device is formed as a suit, to provide at least one support-pad part in the region of a hollow back of the wearer in such a way that the electrode unit bears on the skin of the wearer of the device.

The possibility also exists, however, of disposing two support-pad parts, for example, in the pocket, for example to the right and left of the electrode unit, so that a tensile stress is exerted on the first layer and thus the pressing pressure of the electrode unit on the skin of the wearer is reduced.

In this case, a pressing pressure may be reduced by the support-pad part.

If, for example, an electrode unit is provided in the region of a neck, a lining part may be disposed in such a way in the at least one pocket that a pinpoint pressure exists, so that the electrode unit bears in pinpoint manner or with a small contact area on the body. If, for example, support-pad parts are provided in the border regions of the electrode unit, a pressure relief may be created between the support-pad parts.

This pressure relief may be desirable, for example, in a strength training, in which it is not wished to exert such great pressure with the electrode unit on the shoulders in the neck region that this pressure acts unpleasantly during a strength training.

In other trainings, it is desirable, for example, to allow the at least one electrode unit to bear with a greater pressure on the skin of the wearer even in the shoulder region, in order to achieve an additional good stimulation.

The device according to the invention has not only the advantage that the pressing pressure of the at least one electrode unit may be adjusted individually by the at least one support-pad part, but also the further advantage that the at least one support-pad part may be removed from the at least one pocket of the device and that consequently the device may be easily cleaned, for example washed and sterilized.

Because of the removal of the support-pad parts, these parts may be cleaned and sterilized separately.

They may also be dried separately, so that the device is ready for service again more quickly. This increased readiness is because the support-pad parts usually take longer for drying than the device itself.

In addition, the device according to the invention is very low-priced, because only at least one pocket, in which different support-pad parts may be disposed exchangeably in different numbers, is provided in the device.

No expensive inflatable air cushions or actuators or the like are provided.

Only various support-pad parts adapted individually to the need by the appropriate choice are disposed in the at least one pocket.

According to an advantageous embodiment of the invention, it is provided that the at least one pocket is disposed in such a way on the device that the at least one electrode unit is formed in a manner overlapped at least partly by the at least one pocket.

This embodiment has the advantage that the at least one support-pad part directly exerts a pressing pressure of the electrode unit on the skin of the wearer, because the pocket overlaps the electrode unit at least partly and the support-pad part may be disposed in such a way in the pocket that the electrode unit is pressed directly on the skin of the wearer.

A further advantageous embodiment of the invention provides that the at least one support-pad part is formed from a thermoplastic polymer, from foam, from nonwoven fabric or from silicone, or that the at least one support-pad part is designed as air cushions or spacing knitted fabric.

In the design as air cushions, the air cushions are formed in predefined manner and not inflatably.

According to a particularly preferred embodiment of the invention, the support-pad part consists of a thermoplastic polymer.

In this case, the support-pad part may be manufactured particularly inexpensively as an injection-molded part. In addition, the injection-molded parts may be made in any desired shapes, so that the parts are diversely usable.

Precisely the possibility exists, however, of forming the at least one support-pad part from foam, from nonwoven fabric or from silicone. Likewise, the design as air cushions or spacing knitted fabric is advantageous, because support-pad parts of these materials are able to exert a corresponding pressure on the electrode unit, if they are disposed in the pocket or, as already described, are able to create a pressure relief.

According to an advantageous embodiment of the invention, the at least one pocket has an area that corresponds to the area of the electrode unit.

In this case, it is possible to dispose at least one support-pad part in the pocket in such a way that the electrode unit bears with a homogeneous pressing pressure on the skin of the wearer of the device.

According to another advantageous embodiment of the invention, it is provided that the at least one pocket has a larger or smaller area than the electrode unit.

If the pocket has a larger area than the electrode unit, it is possible, for example, to dispose at least one support-pad part at the border of the electrode unit or outside the electrode unit, so that a pressure relief is achieved.

If the pocket has a smaller area, a higher pressure may be generated in pinpoint manner on the electrode unit with at least one support-pad part.

According to a particularly preferred embodiment of the invention, at least one support-pad part is disposed in the at least one pocket so that the at least one electrode unit is formed to bear with a predetermined pressure on a body of the wearer.

Due to the arrangement of the at least one support-pad part in the at least one pocket, the pressure with which the electrode unit bears on the skin of the wearer may be freely chosen. For example, one or more support-pad parts may be disposed side-by-side or one above the other, relative to a direction orthogonal to the electrode unit in the at least one pocket.

If the pressing pressure is too low, several support-pad parts may be disposed one above the other in the at least one pocket. The possibility also exists of using a support-pad part that has a greater thickness.

According to an advantageous embodiment of the invention, the support-pad parts may have a constant cross section. The possibility also exists of providing a non-constant cross section of the support-pad parts.

According to a further advantageous embodiment of the invention, it is provided that various exchangeable support-pad parts respectively have different thicknesses and/or degrees of hardness.

The support-pad parts may have different thicknesses, so that it is possible to choose a support-pad part that assures the necessary pressing pressure.

The support-pad parts may also have different degrees of hardness, in order to adjust the necessary pressing pressure by means of the different degrees of hardness.

A further advantageous embodiment of the invention provides that the two layers of the device are joined together to at least one pocket by gluing, sewing and/or welding.

The layers are joined together in the border region of the pocket. One side of a pocket remains completely or at least partly open, in order to be able to dispose a support-pad part in the pocket.

The borders of the pocket are advantageously formed by gluing of the two layers, by sewing the two layers together and/or by welding the two layers. The possibility also exists of providing different types of joining.

A pocket is characterized in that at least one opening is provided. Via this opening, the at least one support-pad part may be disposed in the pocket or removed via this opening. A pocket may also have several openings. Hereby, for example, a planar arrangement of a support-pad part is facilitated when a pocket has, for example, two openings disposed opposite one another.

According to a further advantageous embodiment of the invention, at least one additional pocket is disposed outside a region having an electrode unit on the first layer.

Due to these pockets, which are not disposed in the region of an electrode unit, the possibility exists of disposing support-pad parts and herewith achieving a pressure relief.

Pockets that are not disposed in the region of an electrode unit may also be provided, for example on diametrically opposite sides of the suit.

Thus, for example, an electrode unit may be disposed in the region of the lumbar vertebrae and a pocket in the abdominal region. If at least one support-pad part is disposed in the abdominal region, a higher pressing pressure of the suit is generated in the lumbar vertebral region. This type of design is possible when parts of the device or the device itself has a tubular shape, so that a pocket and a corresponding support-pad part in the pocket has a tensile effect on an electrode unit disposed diametrically or almost diametrically opposite the pocket.

According to a particularly preferred embodiment of the invention, the device is designed as a clothing item, especially as a textile suit or as part of a textile suit.

The device is designed particularly advantageously as a suit that consists of a textile material. The suit bears at least partly on the skin of the wearer.

The device may also be designed, however, as part of a textile suit, for example as a sleeve or legging or cuff, as a body or the like.

The pressing pressure is the textile pressing pressure of the device, for example of the textile suit.

The textile substance of the device, for example of the suit, may be designed elastically or non-elastically or as a combination of elastic and non-elastic substance. Also hereby, the pressing pressure of the electrode unit can be adjusted by disposing at least one support-pad part in at least one pocket.

According to a further advantageous embodiment of the invention, the electrode unit has at least one of the following layers:
a) base layer with at least one electrode disposed in or on the base layer, and
b) layer with one of the following layer structures:
b1) plastic layer and
   layer of knitted fabric and
   plastic layer or
b2) layer of knitted fabric and
   plastic layer or
b3) plastic layer and
   layer of knitted fabric
and the layers a) and b) are joined tightly with one another.

The device according to the invention with at least one electrode unit may be used on the one hand for an electrostimulation. The at least one electrode of the electrode unit is energized with current and for this reason may be used for an electrostimulation. It is also possible, however, to perform a data acquisition with the device according to the invention with the at least one electrode unit. For example, a data acquisition may be performed by an EKG (electrocardiogram), an EMG (electromyography) or, the device according to the invention may perform a data acquisition, for example, by measurement of bioimpedances, etc.

The device according to the invention has the following structure with the following layers:

a) base layer with at least one electrode disposed in or on the base layer,
and

b) layer with one of the following layer structures:

b1) plastic layer and
layer of knitted fabric and
plastic layer

or

b2) layer of knitted fabric and
plastic layer

or

b3) plastic layer and
layer of knitted fabric.

In the electrode unit according to the invention, the layers a) and b) are joined tightly with one another. In other words, the layer a) is joined tightly with the layer b1) or the layer a) with the layer b2) or the layer a) with the layer b3). Advantageously, the layers of the layer b1) or of the layer b2) or of the layer b3) are likewise joined tightly with one another.

Due to this structure of the electrode unit, it is ensured that the layer b) has a layer of a knitted fabric. Hereby this layer structure remains stable, even if the base layer with the further layer is changed frequently in its basic shape, for example bent, kinked, stretched, squashed or the like.

The electrode unit may be disposed, for example, on the device according to the invention, for example a suit for an electrostimulation. This suit advantageously contains at least one electrode unit. Depending on arrangement on the suit, the at least one electrode unit is more or less strongly twisted or stretched or squashed or stressed in other ways. Due to configuration of the electrode unit with the layer of knitted fabric with at least one plastic layer, the electrode unit remains stable and functional even over a long usage time, even in case of a strong mechanical stress.

According to an advantageous embodiment of the invention, the electrode is embroidered into the base layer. The embroidery has the advantage that the electrode may be disposed in the desired shape in the base layer. This embroidered electrode is very durable.

According to an advantageous embodiment, the electrode is embroidered with electrically conductive threads into the base layer. Advantageously, the electrically conductive threads are embroidered only into a surface of the base layer. Thus they are not embroidered throughout the entire base layer. Retaining threads that are not electrically conductive may be disposed on the opposite side. In principle, however, the possibility also exists that the electrically conductive threads are disposed continuously through the entire base layer.

According to further advantageous embodiments, the at least one electrode in the base layer may also be disposed in or on the base layer by printing, sewing, gluing and/or inserting and fixing.

The electrode unit is advantageously disposed on only one side of the base layer. In other words, the base layer has the electrically conductive electrode on one side. On the other side, the base layer is not electrically conductive at all or is electrically conductive to only a small extent.

According to a particularly preferred embodiment of the invention, it is provided that the at least one electrode is disposed in or on the prefabricated base layer. This embodiment has the advantage that the electrode may be embroidered in any desired form onto the prefabricated base layer of the electrode unit. The base layer is inherently produced in finished condition. Then the at least one electrode is disposed in or on this prefabricated base layer. If, for example, an adaptation of electrodes to particular body regions is necessary, this adaptation may be defined in the embroidering process.

It is not necessary to define the design of the entire electrode unit from the very beginning, as early as during production of the base layer. The final form in the base layer is defined only during the production of the electrode unit. In this way, electrode units with different designs may be produced inexpensively.

The at least one electrode is advantageously formed from at least one electrically conductive thread. Several electrodes may be produced with one thread or strand of thread. These electrodes are then connected in series. The possibility also exists of producing one or more electrodes from respectively one electrically conductive thread. In this case, one or more electrodes may be connected in parallel. The electrodes may be formed individually or together or in a manner that can be activated in groups.

According to a further advantageous embodiment of the invention, it is provided that the at least one plastic layer is designed to be electrically conductive. Hereby it is possible to achieve a current transmission via the plastic layer.

According to a further advantageous embodiment of the invention, it is provided that the at least one plastic layer is formed from a thermoplastic material and/or from thermoplastic elastomers and/or from conductive silicones.

These materials are particularly flexible and may be deformed together with the suit or a cuff or the like. In addition, the flexibility is preserved over a very long time.

According to a further advantageous embodiment of the invention, it is provided that the at least one plastic layer contains electrically conductive particles. Hereby the at least one plastic layer is likewise electrically conductive. Due to the plastic layer, the user of the inventive device, for example the patient or an athlete, works up a sweat more rapidly. The conductivity is increased by the moisture formed from the sweat.

All plastic layers disposed in the layers b1), b2) or b3) may contain electrically conductive particles. The possibility also exists, however, that only some of the plastic layers disposed in the layers b1), b2) or b3) contain electrically conductive particles.

The electrically conductive particles are advantageously disposed homogeneously or at least approximately homogeneously in the at least one plastic layer.

According to a further advantageous embodiment of the electrode unit, it is provided that the electrically conductive particles consist of silver and/or graphite. These particles can be easily introduced into the plastic layer, and they increase the electrical conductivity of the at least one plastic layer.

A further advantageous embodiment of the electrode unit provides that the base layer consists of a nonwoven fabric. The possibility also exists, however, of using other materials for the base layer. Because the electrode unit is produced by a hot-pressing method, for example, the base layer should be heat-resistant. It should be pressed together, not too strongly, in a thermal pressing method.

A further advantageous embodiment of the electrode unit provides that the at least one plastic layer is formed as polyurethane film. Polyurethane film can be produced particularly inexpensively. In addition, the electrically conductive particles can be disposed well and easily in the polyurethane film.

A particularly preferred embodiment of the electrode unit provides that the at least one layer of knitted fabric contains
gauze and/or
elastic gauze and/or
grid-like material and/or
grid-like elastic material
and/or is formed
from gauze and/or

from elastic gauze and/or
a grid-like material and/or
a grid-like elastic material.

In other words, a layer of gauze and/or of elastic gauze and/or a grid-like material and/or a grid-like elastic material is advantageously formed in the layers b1), b2) or b3).

The layers b1), b2) or b3) may also contain gauze and/or grid-like material, preferably elastic gauze and/or grid-like elastic material.

The gauze and/or the grid-like material are preferably elastic.

The knitted fabric, for example the gauze and/or the grid-like material, preferably elastic material, ensure that the at least one plastic layer does not become brittle or that it withstands higher mechanical loads. This layer of knitted fabric imparts a high stability to the electrode unit with simultaneous flexibility of the electrode unit, so that the electrode unit may be adapted to body parts of the patient or athlete. The knitted fabric, for example the gauze and/or the grid-like material, are advantageously formed elastically in one direction but particularly advantageously in two directions, for example in x direction and in y direction, in order to ensure a best-possible adaptation of the electrode unit to a body part or to the body of a patient or athlete.

Advantageously, the knitted fabric, for example the gauze or the grid-like material, is formed from polyester. This material is inexpensive, durable for a long time and can be formed well as knitted fabric, for example in the form of gauze or grid-like material having the appropriate elasticity.

Advantageously, the electrode unit according to the invention is characterized in that the layers are designed as
layers welded together with one another by a thermal pressing method or
layers joined tightly by a thermal pressing method with a primer or
layers joined tightly with one another by laminating or
layers joined tightly with one another by a high-frequency welding or an ultrasonic welding.

Connecting the various layers tightly with one another by means of the aforesaid methods has the advantage that the method can be performed inexpensively and the flexibility of the electrode unit is nevertheless ensured. In addition, tight, undetachable joints are formed between the layers.

According to a further advantageous embodiment of the electrode unit, it is provided that the electrode unit with the base layer is disposed on a whole body suit or partial body suit. The whole body suit overlaps the central body as well as the extremities, whereas a partial body suit may be designed in the form, for example of a tee shirt, a pullover or a kind of trousers. Due to this design, it is possible, for example for an athlete, to equip a whole body suit with the device according to the invention, so that an electrostimulation, for example, may take place during the sporting activity.

According to another advantageous embodiment of the invention, it is provided that the electrode unit with the base layer is disposed on a cuff. This cuff may be disposed, for example, on the extremities of an athlete or of a patient, in order likewise to perform an electrostimulation during a sporting activity or to perform an electrostimulation for therapeutic purposes or to acquire data, for example in the form of an EKG or EMG.

According to a further advantageous embodiment, it is provided that the device consists of
polyester and elastane or
polyamide 6 or elastane or
polyamide 6.6 and elastane or
polyurethane and elastane.

These materials are particularly advantageous, because the whole or partial body suit or the cuff is configured flexibly hereby, in order to accommodate shearing movements, twisting movements, stretching and/or squashing movements of the whole or partial body suit or of the cuff during movement of the athlete or patient, so that the electrode unit with the at least one electrode remains constantly in body contact.

According to a further advantageous embodiment of the invention, the electrode has a resistance relative to the electrical conductivity of less than 100 Ohm per 10 centimeters of length of the electrode. This feature is advantageous, because the electrodes hereby have a large conductivity, and so an effective electrostimulation may take place.

A particularly preferred embodiment of the device according to the invention with the at least one electrode unit provides that an embroidered electrode is used that is joined with the further layers in a hot-pressing method.

If only one plastic film that contains conductive particles is used, damage occurs easily due to the constant movement of the flat electrode. For this reason, it is advantageous to additionally provide, for example, a knitted fabric in the form of a gauze or of a grid-like elastic material.

The base layer with the electrode is likewise advantageously designed to be low-ohmic, which means that it advantageously has a high conductivity and a very low resistance.

The knitted fabric, for example the gauze, preferably elastic, or the grid-like, preferably elastic material, advantageously consists of polyester or other materials. The gauze is likewise advantageously designed to be elastic.

The at least one plastic layer consists advantageously of a PU (polyurethane) film, which particularly advantageously contains electrically conductive particles, for example graphite and silver. The PU film may also consist of thermoplastic material or of thermoplastic elastomers and also of conductive silicones.

If the athlete is wearing a whole body suit or partial body suit, he or she works up a sweat during the sporting activity. Hereby the base layer, which is disposed in the direction of the skin, is permeated with sweat. Due to the increase of the moisture, the conductivity is also improved.

The plastic layers are advantageously laminated onto the knitted fabric, for example the gauze and/or the grid-like elastic material. The possibility also exists of laminating the electrode unit onto the plastic film in the thermal method.

If the electrode unit is disposed in a whole body or partial body suit, this suit advantageously has, on the outside, a nonconductive layer, for example a layer of polyurethane. The electrode unit may have a joint to the suit, for example by means of gluing or else by means of a thermal method. Particularly advantageously, the plastic films are formed from a thin flexible material, which has a high conductivity, for example with a resistance of less than 100 ohm per 10 cm length of the electrode.

The electrically conductive fibers, which for example are embroidered, inserted or printed in, may consist, for example, of stainless steel or copper.

The at least one support-pad part, which is disposed in the at least one pocket, is advantageously designed in such a way that it generates a homogeneous pressing pressure or a pinpoint pressing pressure of the electrode unit on the skin of the wearer.

As already stated, the at least one support-pad part may also be designed in such a way that it is designed for relief of the pressing pressure of the electrode unit applied by the device. If the at least one support-pad part is provided to exert a pressing pressure, the pressing pressure may be generated in pinpoint or spatial manner.

As already stated, the support-pad parts may have different degrees of hardness. The support-pad parts may also be designed to be rigid.

The textile material of the device, especially of the clothing item, is advantageously designed to be elastic. This elasticity has the advantage that the wearer is able to move very well with the device, especially with the clothing item.

In principle, the possibility also exists that the material of the device, meaning preferably the clothing item, may also be designed to be non-elastic. This non-elasticity may be provided particularly advantageously when the device is designed as a sleeve or legging or cuff.

The possibility also exists of designing the textile material of the clothing item as a combination of elastic and non-elastic material.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a partial body suit with electrode units;

FIG. 2 shows an electrode unit in cross section;

FIG. 3 shows a different example of an electrode unit in cross section;

FIG. 4 shows a different example of an electrode unit in cross section;

FIG. 5 shows a cross section through a first exemplary embodiment of a device according to the invention;

FIG. 6 shows a plan view of the device according to FIG. 5;

FIG. 7 shows a cross section through a second exemplary embodiment of the device according to the invention;

FIG. 8 shows a plan view of the exemplary embodiment of FIG. 7;

FIG. 9 shows a third exemplary embodiment of a device according to the invention in cross section;

FIG. 10 shows a plan view of the device according to the invention according to FIG. 9;

FIG. 11 shows a cross section through a further exemplary embodiment of the device according to the invention; and

FIG. 12 shows a plan view of the exemplary embodiment according to FIG. 11.

### DETAILED DESCRIPTION OF PREFERRED EMBODIMENTS

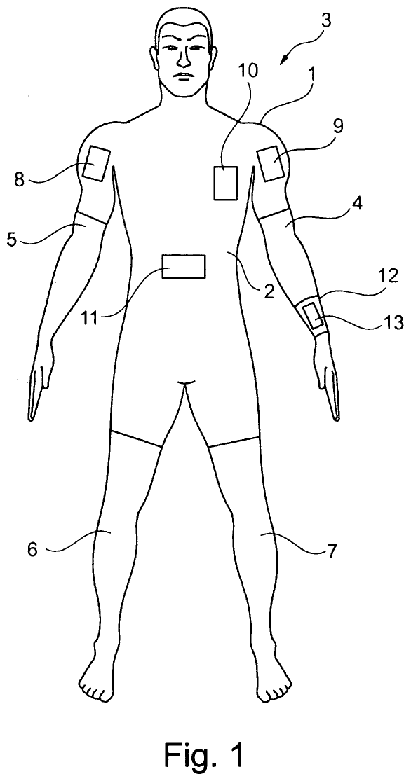

FIG. 1 shows a partial body suit 1, which overlaps a trunk 2 of a person 3. Extremities 4, 5 (arms) and 6, 7 (legs) remain partly free of the partial body suit 1. Electrode units 8, 9, 10, 11 are disposed on the partial body suit 1. The electrode units 8, 9 are disposed in the upper arm region, the electrode 11 in the abdominal region and the electrode 10 in the heart region of the person 3. In addition to those units, the person 3 wears, on the extremity 4, meaning an arm, a cuff 12, on which an electrode unit 13 is disposed. The electrode units 8, 9 and 11 may be used for an electrostimulation. The electrode units 10, 13 may be used, for example, for a data acquisition, for example for an EKG.

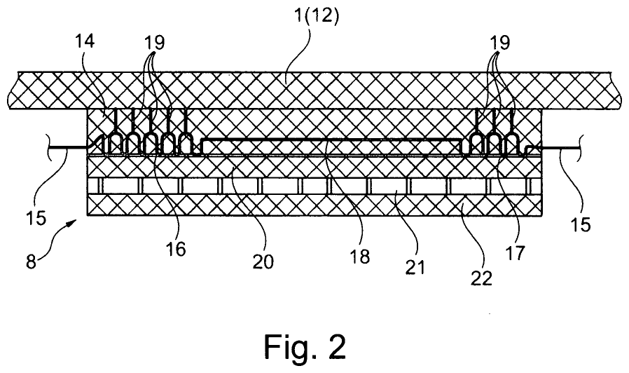

FIG. 2 shows the electrode unit 8, which is disposed on the suit 1 or on the cuff 12. The electrode unit 1 consists of a base layer 14. Two electrodes 16, 17 of an electrically conductive thread 15 are embroidered into the base layer 14. The thread 15 emerges from both sides of the base layer 14, so that an electrical circuit may be formed. Between the electrodes 16, 17, an electrically conductive connection 18 is likewise formed with the thread 15. The thread 15 is embroidered only on the side of the base layer 14 turned away from the suit. The thread 15 is fixed in the base layer 14 with retaining threads 19, which are not electrically conductive. In principle, the possibility also exists that the electrodes 16, 17 are formed from two threads 15, so that two electrical circuits are present. In this case, the electrodes 16, 17 can be activated individually.

The base layer 14 consists of nonwoven fabric and is tightly fastened to the suit 1 or the cuff 12 by means of gluing, thermal pressing methods or the like. A plastic layer 20 is disposed on the side of the base layer turned away from the suit 1 or the cuff 12. A further layer of a knitted fabric, in the present case of a gauze 21, is disposed on the plastic layer 20. In addition, a further plastic layer 22 is provided.

The gauze 21 advantageously consists of an elastic material. It may consist of polyester or other materials.

The plastic films 20, 22 are advantageously formed as polyurethane film. Electrically conductive particles (not illustrated) such as graphite or silver, for example, may be disposed in the plastic layers 20, 21, either in the one layer or in the other layer or in both layers. The plastic layers 20, 22 may also consist of thermoplastic material or of thermoplastic elastomers and also of conductive silicones.

The layers 14, 20, 21, 22 may be joined tightly with one another in a hot-pressing method. All described layers (14, 20, 21, 22) on suit 1 or cuff 12 are joined tightly with one another.

FIG. 2 shows the structure of the layers as described in a first embodiment with the layer structure b1).

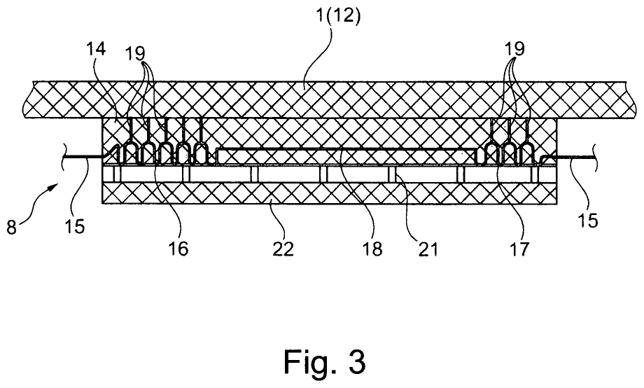

In FIG. 3, like parts as in FIG. 2 are denoted by the same reference symbols. The electrode unit 8 consists of the base layer 14 with the electrodes 16, 17 embroidered therein. The gauze 21 is disposed on the base layer 14 on the side turned away from the suit 1 or the cuff 12. Furthermore, a plastic layer 22 is provided. This structure corresponds to the structure b2) of the first embodiment. The materials are the materials that were described for FIG. 2.

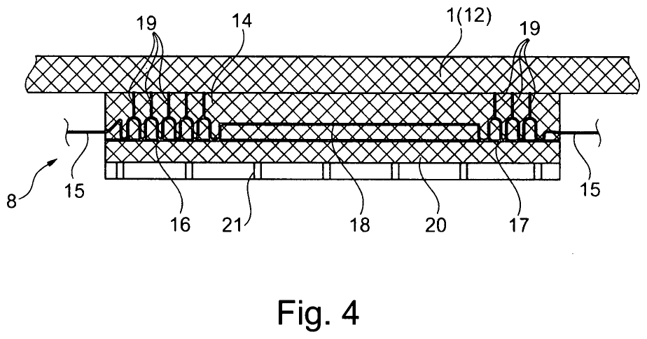

FIG. 4 shows a further possible structure. Like parts as in FIGS. 2 and 3 are denoted by the same reference symbols. The base layer 14 with the electrodes 16, 17 is disposed on the suit 1 or the cuff 12. The plastic layer 20 is disposed on the side of the base layer 14 turned away from the suit 1 or the cuff 12. The gauze 21 is disposed on the plastic layer 20. This structure corresponds to the structure b3) of the first embodiment.

The base layer 14 may consist of a nonwoven fabric, a knitted fabric, a warp-knitted fabric or the like. The electrodes 16, 17 are advantageously embroidered into the base layer 14. They may also, however, be printed, inserted, tightly glued or the like.

Due to the arrangement of the layer structure on the base layer on the side that is turned away from the suit or the cuff with at least one plastic layer 20, 22 and the gauze 21, a flexible electrode unit 8 is formed that is also durable for a long time even during long use with recurring shearing and/or twisting forces and/or stretching and/or squashing.

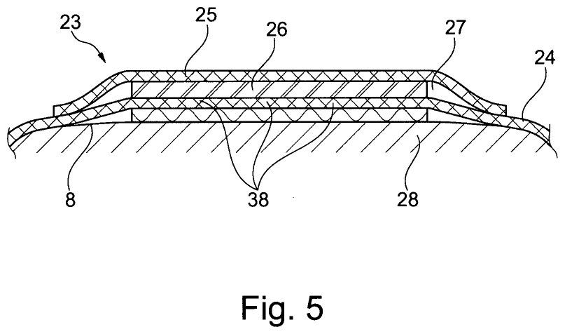

FIG. 5 shows a cross section through a device 23 according to the invention, which in the present case, for example, is designed as a suit. A first layer 24 of the device 23 carries an electrode unit 8, which is designed in a manner bearing on a skin 28 of a wearer (not illustrated in FIG. 5). The device 23 has a second layer 25, which together with the first layer 24 forms a pocket 27. A support-pad part 26 is disposed in the pocket 27. The support-pad part consists, for example, of thermoplastic polymer and may be formed as an injection-molded part.

Due to the arrangement of the support-pad part 26, a region 38 with an increased pressing pressure is formed. The support pad 26 fills the pocket 27 almost completely and the support-pad part 26 has a constant thickness and a constant degree of hardness, so that a homogeneous pressing pressure in the region 38 is produced. Thus, the electrode unit 8 is pressed with a homogeneous pressing pressure on the skin 28.

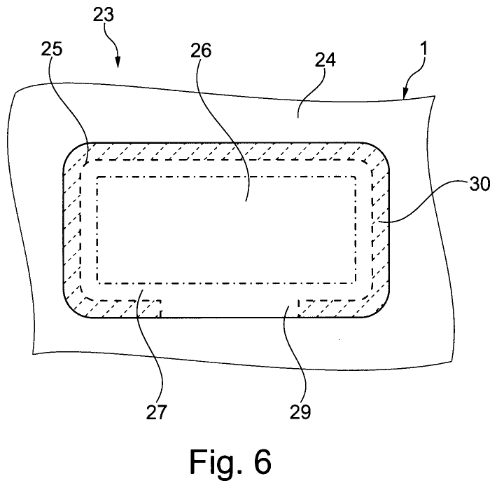

FIG. 6 shows the device 23, for example a partial region of a suit 1. The device 23 has the first layer 24. A second layer 25 forms a pocket 27 together with the first layer 24. The first layer 24 is glued together with the second layer 25 in a region 30. Hereby the pocket 27 is formed. The pocket 27 has an opening 29, via which the support-pad part 26 may be disposed in the pocket 27. The pocket 27 is illustrated by a dash line and the support-pad part 26 is illustrated by a dot-dash line.

The support-pad part 26 may be formed as a relatively soft support-pad part 26. Hereby the homogeneity of the pressing pressure in the region 38 is achieved even better.

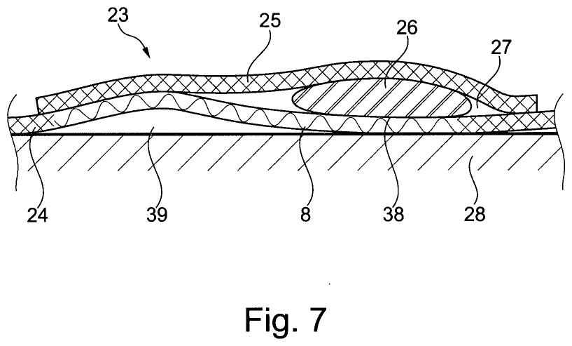

FIG. 7 shows a further exemplary embodiment of the device 23, for example of a body. The device 23 has a first layer 24, in which, according to FIG. 7, the electrode unit 8 is disposed in integrated manner. Together with the second layer 25, the first layer 24 forms a pocket 27, in which the support-pad part 26 is disposed. The support-pad part 26 does not fill the entire pocket 27. For this reason, the electrode unit 8 is pressed in the region 38 with an increased pressing pressure on the skin 28. In the region 39, a relief of the pressing pressure exists, so that the electrode unit 8 in this region bears on the skin 28 not at all or with only a small pressing pressure.

This effect may be further increased by forming the support-pad part 26 as a hard support-pad part 26. Hereby the pressing pressure in the region 38 is also considerably increased yet again.

A relief of the pressing pressure in the region 39 occurs because the support-pad part 26 generates an increased pressing pressure of the electrode unit in the region 38 and thus the pressing pressure is reduced in the region 39.

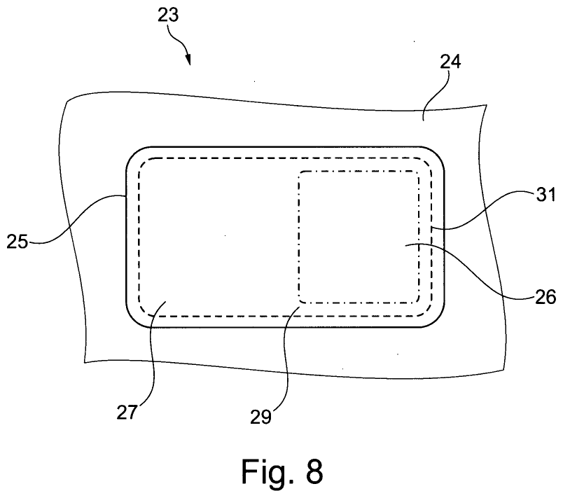

According to FIG. 8, the first layer 24 forms a pocket 27 together with the second layer 25. The first layer 24 and the second layer 25 are sewn together with one another. A seam 31 is provided. No seam 31 is provided in the region 29 of the pocket 27. As illustrated in FIG. 8, the support-pad part 26 is disposed only in a partial area of the pocket 27, as is also illustrated in FIG. 7.

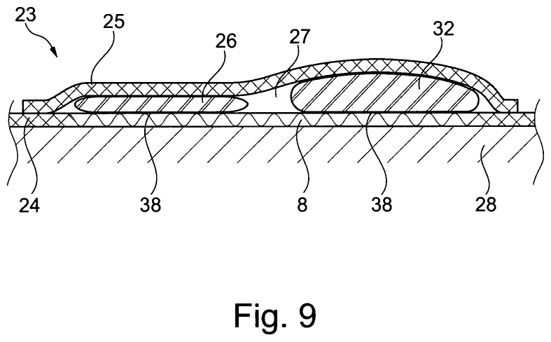

FIG. 9 shows the device 23, in which a first layer 24 together with the second layer 25 forms a pocket 27. The electrode unit 8 is disposed in integrated manner in the first layer 24, so that the electrode unit 8 bears on the skin 28.

Support-pad parts 26, 32 are disposed in the pocket 27, so that two regions 38 with an increased pressing pressure are formed. The support-pad part 32 has a greater thickness than the support-pad part 26. Thus a greater pressing pressure is generated by the support-pad part 32 than by the support-pad part 26.

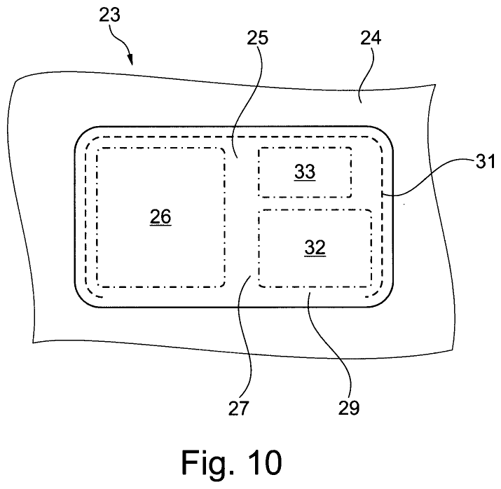

FIG. 10 shows the device 23 of FIG. 9.

The first layer 24 is joined together with the second layer 25 by means of a seam to form a pocket 27. The pocket 27 has an opening 29. The support-pad parts 26, 32, 33 are disposed in the pocket 27. As illustrated in FIG. 9, the support-pad parts 26, 32 have different thicknesses, so that the pressing pressure of the electrode unit 8 (illustrated in FIG. 9) may be of different magnitude.

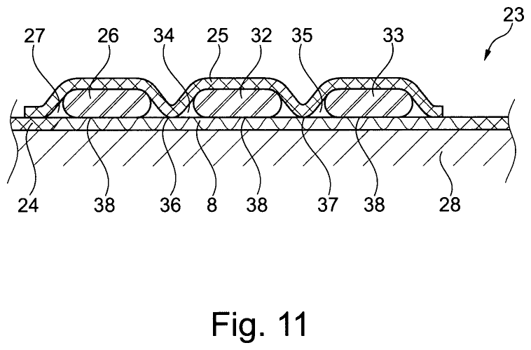

FIG. 11 shows the device 23 with the first layer 24, in which the electrode unit 8 is disposed. The first layer 24 forms three pockets 27, 34, 35 together with the second layer 25. In the regions 36, 37, additional joints between the first layer 24 and the second layer 25 are provided in addition to the joint in the border regions. The joints may be produced by gluing, sewing, welding or the like.

A support-pad part 26 is disposed in the pocket 27, a support-pad part 32 is disposed in the pocket 34 and a support-pad part 33 is disposed in the pocket 35. In the regions 38, an increased pressing pressure of the electrode unit 8 on the skin 28 is generated by the support-pad parts 26, 32, 33.

As illustrated in FIG. 11, the pocket 27 overlaps the left region of the electrode unit, while the pocket 35 incompletely overlaps the right region of the electrode unit 8

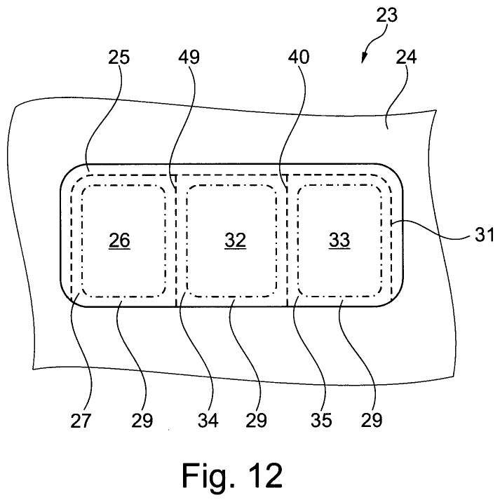

FIG. 12 shows the device 23 according to FIG. 11 with the three pockets 27, 34, 35. The pockets 27, 34, 35 are separated from one another by additional seams 40, 49. The seams 40 form a subdivision of the pocket 27 illustrated in FIGS. 5 to 10.

The seam 31 is provided in order to form the pockets 27, 34, 35. Openings 29 of the pockets 27, 34, 35 remain, in order to dispose the support-pad parts 26, 32, 33 in the pockets 27, 34, 35 and to remove them therefrom.

The illustrated exemplary embodiments of FIGS. 5 to 12 may be combined with one another in any desired manner.

Although only a few embodiments of the present invention have been shown and described, it is to be understood that many changes and modifications may be made thereunto without departing from the spirit and scope of the invention.

What is claimed is:

1. A device formed from a textile material for electrostimulation or for data acquisition by diagnostic instruments comprising:
a first layer;
at least one electrode unit disposed in or on the first layer;
a second layer disposed on the first layer so that the first layer and the second layer form a pocket; and
at least one support-pad part removably disposed in the pocket.

2. The device according to claim 1, wherein the pocket is disposed on the device so that the at least one electrode unit is overlapped at least partly by the pocket.

3. The device according to claim 1, wherein the at least one support-pad part comprises a thermoplastic polymer, a foam, a nonwoven fabric or a silicone material, or wherein the at least one support-pad part comprises air cushions or spacing knitted fabric.

4. The device according to claim 1, wherein the pocket has a pocket area corresponding to an electrode unit area of the at least one electrode unit.

5. The device according to claim 1, wherein the pocket has pocket area larger than an electrode unit area of the at least one electrode unit.

6. The device according to claim 1, wherein the pocket has pocket area smaller than an electrode unit area of the at least one electrode unit.

7. The device according to claim 1, wherein at least one support-pad part is disposed in the pocket so that the at least one electrode unit bears with a predetermined pressure on skin of a person.

8. The device according to claim 1, comprising a plurality of support-pad parts, each support-pad part having a different thickness, degree of hardness, or thickness and degree of hardness.

9. The device according to claim 1, wherein the first and second layers are joined together to the pocket by at least one of gluing, sewing, and welding.

10. The device according to claim 1, wherein the pocket has at least one subdivision.

11. The device according to claim 1, wherein further comprising at least one additional pocket disposed outside a region on the first layer having the at least one electrode unit.

12. The device according to claim 1, wherein the device is designed as a clothing item.

13. The device according to claim 12, wherein the clothing item is or is part of a textile suit.

14. The device according to claim 1, wherein the at least one electrode unit comprises:
layer a) comprising a base layer with at least one electrode disposed in or on the base layer, and layer b) comprising a layer having a layer structure comprising:
b1) a first plastic layer, a knitted fabric layer, and a second plastic layer;
or
b2) the knotted fabric layer and the second plastic layer;
or
b3) the first plastic layer and the knitted fabric layer;
wherein the layers a) and b) are joined with one another, and wherein the base layer is identical with or is part of the first layer.

15. The device according to claim 14, wherein the at least one electrode is disposed in or on the base layer by embroidering, printing, sewing, gluing and/or inserting and fixing.

16. The device according to claim 14, wherein the base layer is a prefabricated base layer and the at least one electrode is disposed in or on the prefabricated base layer.

17. The device according to claim 14, wherein at least one of the first and second plastic layers is electrically conductive.

18. The device according to claim 14, wherein the knitted fabric layer is formed from polyester.

19. The device according to claim 14, wherein the knitted fabric layer contains or is formed from:
(a) gauze and/or
(b) elastic gauze and/or
(c) grid-shaped material and/or
(d) grid-shaped elastic material.
